(19) United States
(12) Patent Application Publication
(10) Pub. No.: US 2024/0289592 A1
(43) Pub. Date: Aug. 29, 2024

(54) Computer Implemented Classification Tool And Method For Classification Of Microelectrode Recordings Taken During A Deep Brain Stimulation

(71) Applicant: Naukowa i Akademicka Siec Komputerowa – Panstwowy Instytut Badawczy, Warszawa (PL)

(72) Inventor: Konrad CIECIERSKI, Warszawa (PL)

(21) Appl. No.: 18/412,819

(22) Filed: Jan. 15, 2024

(30) Foreign Application Priority Data
Feb. 27, 2023 (EP) 23461523.5

(51) Int. Cl. G06N 3/0464; A61B 5/383

(52) U.S. Cl. CPC G06N 3/0464; A61B 5/383

(57) ABSTRACT

A method of creating a computer implemented classification tool for classification of microelectrode recordings taken during a deep brain stimulation using deep residual neural network with attention comprising steps collecting a data set of recordings taken during a deep brain stimulation; splitting recordings into overlapping time chunks, and converting time chunks into spectrograms; dividing data set into a training set, a validation set, and a test set putting each spectrogram into a deep neural network of ResNet architecture augmented with a self-attention layer added after each of ResNet layers, with a head layer comprising a single 2D convolutional layer followed by batch normalization and ReLU activation function wherein the network is trained to return zero for time chunks taken from recordings made outside of the STN region of a brain and to return one for time chunks taken form recordings made within the STN region of a brain, fine tuning the network with the validation set, cross checking the network with the test set.

### BACKGROUND

An aspect of this disclosure is a computer implemented classification tool and method for classification of microelectrode recordings taken during a deep brain stimulation.

During the Deep Brain Stimulation (DBS) surgery for Parkinson's disease (PD), the main goal is to place the permanent stimulating electrode into an area of the brain that becomes pathologically hyperactive. This area, called Subthalamic Nucleus (STN), is small and located deep within the brain. Therefore, the main challenge is precise localisation of the STN region. In this work, there is described a neural network acting as decision support system for the neurosurgical DBS surgery.

Expected target positioning of the stimulating electrode is firstly calculated off-line based on the Computed Tomography (CT) and Magnetic Resonance Imaging (MRI) scans.

Unfortunately, based on these data, it is not possible to accurately discriminate the STN area from the surrounding brain tissue. CT and MRI scans give only the approximate location. Precise localization is calculated on-line (during the surgery) based on measurements of the electrical activity of brain tissue recorded by thin microelectrodes.

WO2009002072A2 discloses a method of determining the position of a deep brain stimulation (DBS) electrode which finds the position of the DBS electrode with respect to a deep brain target region, by using a first volume data set containing information on the deep brain target region and a second volume data set containing information on the DBS electrode implanted toward the deep brain target region, and which includes: a first step of generating a subvolume of the deep brain target region from the first volume data set, and also generating a subvolume of the DBS electrode from the second volume data set; and a second step of overlapping and displaying the subvolume of the deep brain target region and the subvolume of the DBS electrode.

WO2020261282A1 discloses a method for determining position of an electrode lead inside a body tissue, including: receiving electrical signals recorded from at least one macro electrode contact of an electrode lead positioned inside a body tissue; extracting spiking (SPK) signals from the received electrical signals; providing stored measurements or indications thereof; determining a position of the lead and/or the at least one macro electrode contact inside said body tissue based on the extracted SPK signals and the provided stored measurements or indications thereof.

EP1396233A1 discloses an apparatus for use in a brain of a subject is provided, including an instrument, adapted to be inserted into the brain. A set of one or more electrodes, coupled to the instrument, are adapted to sense electrical activity of the brain and to transmit an electrical activity signal responsive thereto. A location sensor, adapted to be coupled to the instrument transmits a location signal indicative of a location of the instrument. A control unit, analyzes the electrical activity signal and the location signal. The control unit determines, responsive to the analysis, a position of the instrument with respect to an image of the brain, and electrophysiological information regarding tissue at the position.

### SUMMARY

The present disclosure introduces a method of creating a computer implemented classification tool for classification of microelectrode recordings taken during a deep brain stimulation using deep residual neural network with attention comprising steps collecting a data set of recordings taken during a deep brain stimulation; splitting recordings into overlapping time chunks, and converting time chunks into spectrograms; dividing data set into a training set, a validation set, and a test set. The method according the disclosure is characterized by the steps of putting each spectrogram into a deep neural network of ResNet architecture augmented with a self-attention layer added after ResNet layers, with a head layer comprising a single 2D convolutional layer followed by batch normalization and ReLU activation function. In this method the network is trained to return zero for time chunks taken from recordings made outside of the STN region of a brain and to return one for time chunks taken form recordings made within the STN region of a brain, fine tuning the network training with the validation set, cross checking the network with the test set.

The method according to the disclosure is characterized in that the collecting data set includes signal recordings of 10 seconds.

The method according to the disclosure is characterized in that the recordings are split into overlapping 500 ms long time chunks.

The method according to the disclosure is characterized in that each spectrogram has dimensions 129 (frequency) by 53 (time).

The method according to the disclosure is characterized in that the 2D convolutional layer has a kernel size 7×7, padding 3 and 64 filters.

The method according to the disclosure is characterized in that each ResNet layer consists of six blocks, one lead-in block and five consecutive proper blocks.

The method according to the disclosure is characterized in that the convolutional layers in ResNet blocks has kernel size 3×3 and padding 1.

The present disclosure also provides a computer implemented classification tool for classification of microelectrode recordings taken during a deep brain stimulation, comprising an input block for receiving signals from microelectrodes in STN region of a brain; a conversion block for converting signals from the time domain into spectrograms; a computer implemented a deep residual neural network with attention created as in any of claims from 1 to 7 for receiving spectrograms. Further it comprises an output block presenting the result of classification done by the deep residual neural network with attention.

The present disclosure also introduces a method of classification of microelectrode recordings taken during a deep brain stimulation using deep residual neural network with attention comprising steps of receiving signals from microelectrodes placed in the STN region of a brain in an input block; converting signals from the time domain into spectrograms in a conversion block; providing spectrograms to a computer implemented a deep residual neural network with attention created as in any of claims from 1 to 7 for receiving spectrograms, and presenting the result of classification done by the deep residual neural network with attention in an output block.

Present disclosure advantageously increase the probability of correct identification of the area within the brain tissue that is suitable for electrostimulation. Signaling from the disclosure provides additional source of information to the neurosurgeon in respect of the functional placing of electrodes i.e. it helps to identify the best possible place to insert an electrode during Deep Brain Stimulation surgery for Parkinson's disease.

The attentional mechanism occurring after ResNet blocks is crucial in this solution. This mechanism intelligently modifies the output of ResNet blocks so that, in effect, it modifies the error signals or interference so that the quality of the classification that the network performs is the best possible. The modification mechanism can both reduce the amplitude of unwanted interference and amplify the amplitude of the signal in selected areas.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure is described in more detail below in reference to the preferred embodiments shown in the drawings, where:

FIG. 1 shows an example of a brain scan with electrodes inserted;

FIG. 2 shows the schematic architecture of the ResNet architecture that has been augmented with a self-attention layer added after each of the ResNet layers according to the preferred embodiment of the disclosure;

FIG. 3 shows ResNet blocks;

FIG. 4 shows a recording with selected chunk containing artifact;

FIG. 5 shows an amplitude of selected chunk;

FIG. 6 shows a spectrogram of selected chunk;

FIG. 7 shows Channel 0;

FIG. 8 shows Channel 5;

FIG. 9 shows Channel 12;

FIG. 10 shows Channel 15;

FIG. 11 shows a schematic of the attention layer;

FIG. 12 shows an attention acting on tensor;

FIG. 13 shows ResNet layer output #1;

FIG. 14 shows ResNet layer output #2;

FIG. 15 shows attention vector #1;

FIG. 16 shows attention vector #2;

FIG. 17 shows ResNet layer output #2 after attention;

FIG. 18 shows ResNet layer output #1 after attention;

FIG. 19 shows ROC curve for chunks;

FIG. 20 shows ROC curve for recordings.

### DETAILED DESCRIPTION

Expected target positioning of the stimulating electrode is firstly calculated off-line based on the Computed Tomography (CT) and Magnetic Resonance Imaging (MRI) scans. Unfortunately, based on these data, it is not possible to accurately discriminate the STN area from the surrounding brain tissue. CT and MRI scans give only the approximate location. Precise localization is calculated on-line (during the surgery) based on measurements of the electrical activity of brain tissue recorded by thin microelectrodes.

A set of parallel recording microelectrodes is inserted into the brain and advanced in measured steps towards the expected location of the STN. Typically three to five recording microelectrodes are used for each brain hemisphere. The STN area is localized, and the coordinates of the stimulating electrode in the stereotactic surgical space is obtained. Unfortunately, the recorded data are adversely affected by various artifacts that can cause false-positive detection of the STN. Therefore, these artifacts must either be removed in data pre-processing or be properly addressed in the decision process.

By analyzing of the recordings, the computer classifier assesses if they were registered within the STN or not. The recordings' classification results provide a 3D localization of the Subthalamic Nucleus (STN) in the stereotactic surgical space. Microelectrode recordings contain various artifacts that can cause false-positive detection of the STN. These artifacts must either be removed in data pre-processing or be properly addressed by the classification algorithm.

Existing solutions are very sensitive to artifacts in the recordings and require complex data pre-processing to remove them. The described neural network-based approach does not require any prior removal of the artifacts.

The implementation of described network provides a novel method for calculating the expected position of the stimulating electrode based on the measurements of electrical activity of brain tissue. The neural network with attention is used during surgery to classify the microelectrode recordings and determine final position of the stimulating electrode within the STN area.

The test were carried on a number of data collected during DBS surgeries, giving encouraging results. The experimental results demonstrate that deep learning methods augmented with self-attention blocks are competing to the other solutions. They provide significant robustness to measurement uncertainty and improve an accuracy of the stimulating electrode placement.

This work describes an approach to classify the recordings using the residual neural network with attention in the temporal domain. The residual neural network has been augmented by including the self-attention blocks between the residual blocks. Obtained results show that attention acting along the temporal domain diminishes the influence of the artifacts. It is also evident that attention has improved the learning process and classification results.

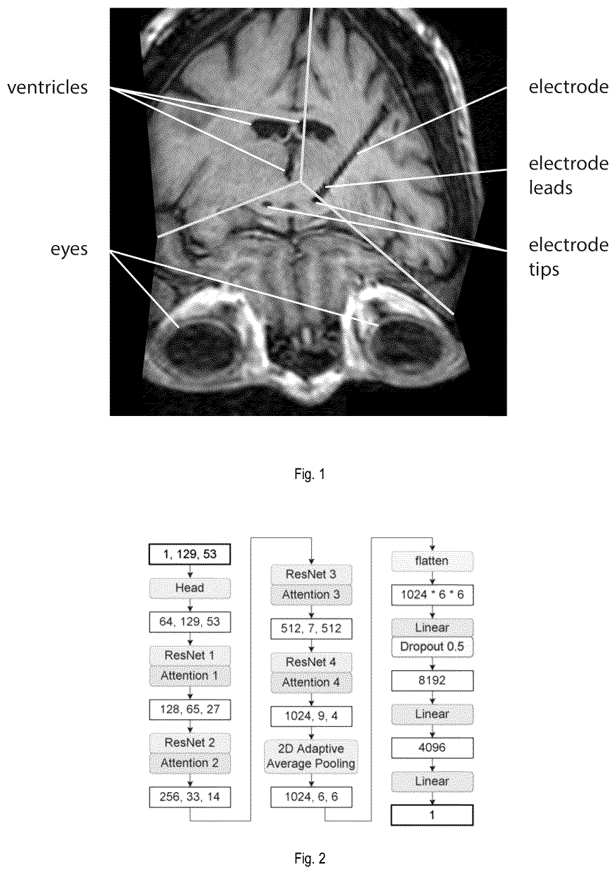

FIG. 1 shows an example of a brain scan with electrodes inserted. The leads of the electrodes are located in the STN, dorsal to the axial plane traversing the eyes. Yellow lines separate the cut-out planes used for visualization. It can be readily seen how deeply the STN is located and how difficult and, at the same time, crucial it is to determine its stereotactic position with maximal precision.

In the progression of the PD disease due to degenerative changes in SNr, the STN becomes hyperactive. This change presents itself in recordings as a higher volume of background noise and increased power of the recorded signals in certain frequency bands. Unfortunately, artifacts present in microelectrode recordings also increase such attributes. If not adequately addressed, this, in turn, leads to false-positive errors, i.e., recordings made outside of the STN having wrong labels pointing to the STN, all because of the presence of the artifacts. In the case of a standard approach to this problem artifacts are detected and removed prior calculation of the classification attributes.

In the solution described in this paper, for successful classification, the network architecture addresses the problem of artifacts.

Analyzed recording is split into overlapping 500 ms long chunks for classification. These chunks are then converted into spectrograms using the Python package SciPy. Each spectrogram has dimensions 129 (frequency) by 53 (time). Spectrograms are then input to the neural network and are classified individually.

The described network is based upon the ResNet architecture that has been augmented with a self-attention layer added after each of the ResNet layers. Schematic architecture is shown in FIG. 2.

Boxes with a white background show the shape of the passing tensor. The shape of the tensor is given in PyTorch notation, i.e., Channels, Height, and Width. For simpler notation, the batch dimension has been omitted on all figures. The network was trained to return zero for chunks taken from recordings made outside of the STN and one for chunks taken from recordings made within the STN.

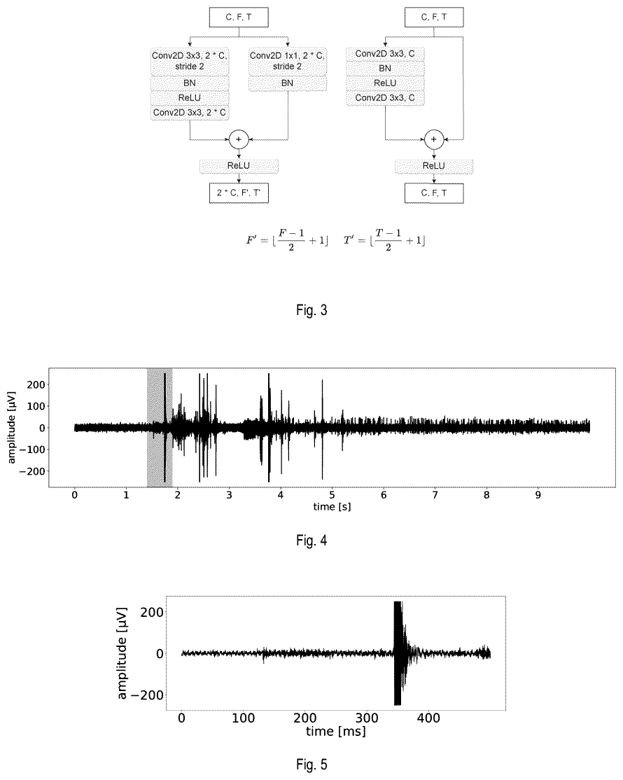

The Head layer comprises a single 2D Convolutional layer followed by Batch Normalization and ReLU activation function. The Convolutional layer has kernel size 7×7, padding 3 and 64 filters. Each Resnet layer consists of six blocks, one lead-in block (shown on the left), and five consecutive proper blocks (shown on the right). All convolutional layers in ResNet blocks use kernel size 3×3 and padding 1. ResNet blocks are shown in FIG. 3.

### Attention in Temporal Domain

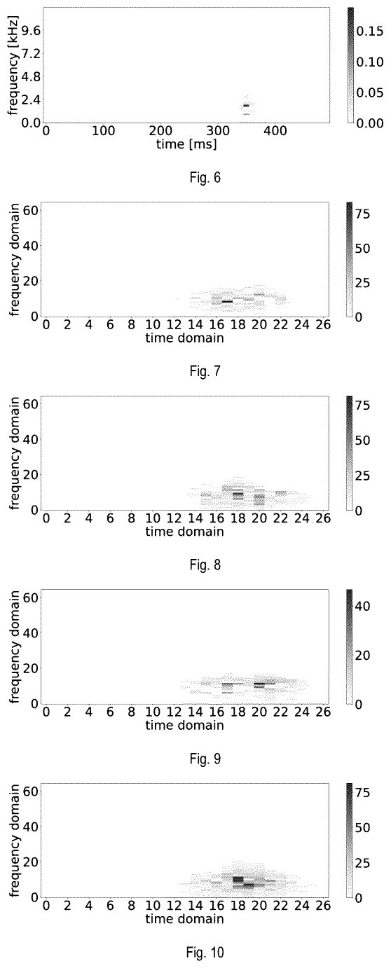

As can be seen in FIG. 4-FIG. 6, the artifact present in the amplitude of the recorded signal (FIG. 4 and FIG. 6) is strongly present not only in the spectrogram spanning selected time period (FIG. 6) but also across the channels in the output of the ResNet block (FIG. 7-FIG. 10).

As channels are computed using different convolutional filters, in some of the channels the artifact is more pronounced than in others (compare FIG. 7 and FIG. 9). Also location of the artifact in the time domain shifts slightly between channels (see FIG. 7 and FIG. 10).

In such a situation, the assumption is that mechanism that would diminish the results of the artifact in the outputs of the ResNet layers should improve the classification results as well as the network training process.

As can be seen in FIG. 7-FIG. 10, ResNet layer output at different channels may differ. Because of this, attention acting in the time domain, i.e., detecting and mitigating results of the input artifact, must be fine-tuned for each channel separately.

Having output of the ResNet layer as tensor with shape C×F×T corresponding to (Channels, Frequency, Time), each time slice can be treated by the attention process separately. Having time slice with shape C×F×1 a dedicated neural network returns a vector of size C×1 that after being expanded to C×F×1 shape is positionally multiplied with the input time slice.

In this way, each channel across all frequencies is multiplied by the calculated channel-specific attention value for each time slice. Use of sigmoid activation function guarantees that attention value stays in (0,1) range.

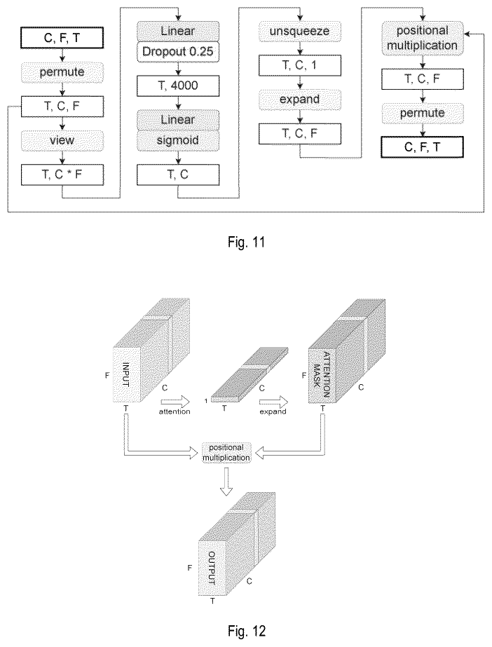

In PyTorch implementation, all time slices are, of course processed in parallel, as it is shown in FIG. 11.

### Experiments

Network has been trained using real medical data obtained during 46 surgeries. During these surgeries 4650 recordings sampled with 24 kHz were registered. 3210 (69%) were labelled as recorded outside of the STN, while 1440 (31%) were labelled as recorded within the STN.

Training of the neural network has been done using Tesla v100 GPU using Python 3.8 with CUDA version 10.1, PyTorch library 1.8.1 and Ignite library 0.4.1.

Recordings were divided in a stratified way into disjoint training, validation, and test sets in proportion 8:1:1 accordingly. 80% of STN recordings and 80% of non-STN recordings were put into the training set. Half of the remaining STN recordings and half of the remaining non-STN recordings were put into the validation set. All remaining STN and non-STN recordings were put into the test set.

The recordings in each of the sets were then divided into overlapping chunks. Chunks of the length 12 000 samples (i.e. 500 ms) were taken with step 2 400 samples (i.e. 100 ms). In this way, a recording lasting 10 s yielded 96 chunks, each with a length of 0.5 s. The division into overlapping chunks acts as an augmentation and reduces the volume of the data put through the neural network at a given time.

After the augmentation process, a set of 412 348 chunks was obtained; there were 330 348 training chunks, 41 040 validation chunks, and 40 960 test chunks. As explained in scientific literature the most informative data for the localization of the STN resides in the frequency domain. For this reason, each chunk has been transformed into its spectrogram using the spectrogram method from the Python package SciPy. The window used for generating spectrograms was Tukey with shape parameter 0.25 and length 256. As a result, each chunk produces a spectrogram with dimensions 129 (frequency) by 53 (time), see FIG. 2.

During the training, the cyclic learning rate has been applied with boundaries between 1e-8 and 1e-7. Cycle length has been set to match the length of an epoch. The stop condition for training was set to trigger after 25 consecutive epochs without a decrease of the loss value calculated for validation data.

The attention mechanism can also be visualized as calculation of T×C×1 attention tensor for input tensor of shape T×C×F. The attention tensor is then expanded to T×C×F shape and positionally multiplied with input to obtain the output tensor (see FIG. 12).

One can notice on FIG. 12 that for selected channel slice, there is an calculated attention vector of size T. This vector is then positionally multiplied across all frequencies of the selected channel slice. As the attention uses the sigmoid activation function, values of this vector are in the (0,1) range.

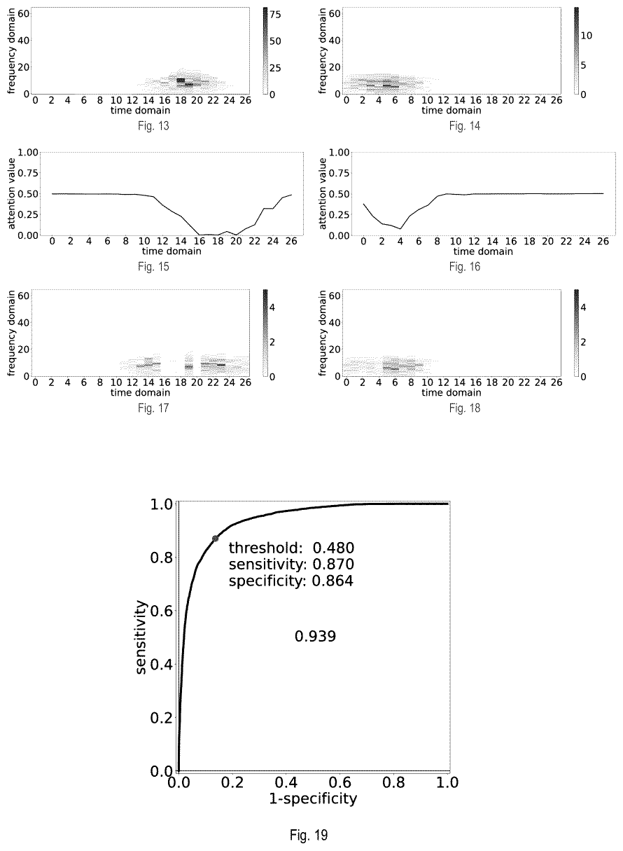

FIG. 15 and FIG. 16 show the values of the attention vector calculated by the trained network for selected ResNet layer outputs. One can readily see that for time ranges where the artifact is present, the value in the attention vector drops to mitigate it. It is also evident that for artifact with high amplitude (see FIG. 13, time index 16-20) the value of the attention vector (see FIG. 15, time index 16-20) is much smaller than in case of moderate artifact (see FIG. 14 and FIG. 16). FIG. 13 and FIG. 14 show data that can be visualized as highlighted slice in INPUT in FIG. 12. FIG. 17 and FIG. 18 show data that can be visualized as highlighted slice in OUTPUT in FIG. 12.

Experiments shown that constructed attention mechanism is working. The attention mechanism emerges in an unsupervised way during the training, and it can work both for the diminishing of the artifacts and as a booster for other parts of the data passing through the neural network.

Finally, trained network was used for classification of the recordings from the training set of test set of 40 960 test chunks. The obtained AUC is 0.939 and the red point closest to upper-left corner (FIG. 19) denotes sensitivity 0.870 and specificity 0.864. The classification threshold for the red point 0.48.

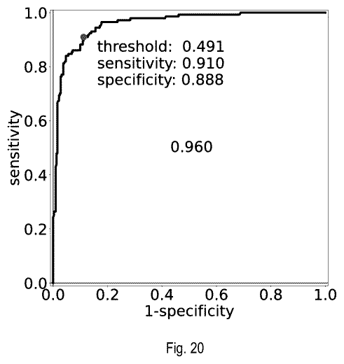

Classification of recordings is based upon the classification of the chunks obtained from them. The classification value for recording is a mean of results returned by the network for its chunks. The AUC for recordings (see FIG. 20) is 0.96. The red point closest to the upper-left corner denotes the sensitivity of 0.91 and specificity of 0.888. The classification threshold for the above results is 0.491.

Classifier based upon described architecture achieved for chunks the AUC value above 0.9 which according to scientific literature is considered outstanding. In case of classification of the recording the AUC value is even higher i.e., above 0.95.

1. A method of creating a computer implemented classification tool for classification of microelectrode recordings taken during a deep brain stimulation using deep residual neural network with attention, comprising steps of:
collecting a data set of recordings taken during a deep brain stimulation;
splitting recordings into overlapping time chunks, and converting time chunks into spectrograms;
dividing data set into a training set, a validation set, and a test set putting each spectrogram into a deep neural network of ResNet architecture augmented with a self-attention layer added after each of ResNet layers, with a head layer comprising a single 2D convolutional layer followed by batch normalization and ReLU activation function wherein
the network is trained to return zero for time chunks taken from recordings made outside of the STN region of a brain and to return one for time chunks taken form recordings made within the STN region of a brain,
fine tuning the network with the validation set,
cross checking the network with the test set.

2. The method of claim 1, wherein collecting data set includes signal recordings of 10 seconds.

3. The method of claim 1, wherein the recordings are split into overlapping 500 ms long time chunks.

4. The method of claim 1, wherein each spectrogram has dimensions 129 (frequency) by 53 (time).

5. The method of claim 1, wherein the 2D convolutional layer has a kernel size 7×7, padding 3 and 64 filters.

6. The method of claim 1, wherein each ResNet layer consists of six blocks, one lead-in block and five consecutive proper blocks.

7. The method of claim 1, wherein the convolutional layers in ResNet blocks has kernel size 3×3 and padding 1.

8. A computer implemented classification tool for classification of microelectrode recordings taken during a deep brain stimulation, comprising:
an input block for receiving signals from microelectrodes in STN region of a brain;
a conversion block for converting signals from the time domain into spectrograms;
a computer implemented a deep residual neural network with attention created according to claim 1 for receiving spectrograms
an output block presenting the result of classification done by the deep residual neural network with attention.

9. A method of classification of microelectrode recordings taken during a deep brain stimulation using deep residual neural network with attention, comprising steps of:
receiving signals from microelectrodes placed in the STN region of a brain in an input block;
converting signals from the time domain into spectrograms in a conversion block;
providing spectrograms to a computer implemented a deep residual neural network with attention created according to claim 1 for receiving spectrograms
presenting the result of classification done by the deep residual neural network with attention in an output block.
